(19) United States
(12) Patent Application Publication
(10) Pub. No.: US 2025/0263079 A1
(43) Pub. Date: Aug. 21, 2025

(54) INTEGRATED USER HEALTH AND WELLNESS MANAGEMENT

(71) Applicant: TOYOTA MOTOR ENGINEERING & MANUFACTURING NORTH AMERICA, INC., PLANO, TX (US)

(72) Inventor: Pujitha GUNARATNE, Northville, MI (US)

(73) Assignees: TOYOTA MOTOR ENGINEERING & MANUFACTURING NORTH AMERICA, INC., PLANO, TX (US); TOYOTA JIDOSHA KABUSHIKI KAISHA, TOYOTA-SHI (JP)

(21) Appl. No.: 18/583,710

(22) Filed: Feb. 21, 2024

(51) Int. Cl. B60W 40/08

(52) U.S. Cl. CPC B60W 40/08; B60W 2040/0872

(57) ABSTRACT

A wellness learning platform may collect and analyze heterogenous data streams representative of multiple individualized behavioral and physiological data/parameters or characteristics of users or subjects, such as vehicle drivers. Such parameters can be observed from the users' own actions or physiology/physiological response(s), as well as from “user-adjacent” behaviors or conditions observed, e.g., from the way users operate a vehicle or interact with the users' environment(s). The parameters can then be used to train personalized models (generated using, for example, a digital twin system or machine-learning (ML)/artificial intelligence (AI) mechanisms with which the collection/analytical platform is operatively connected) to predict the onset of disease conditions. Notifications suggesting remediating actions or instructions in response to identifying some disease onset may be provided to the user.

### TECHNICAL FIELD

The present disclosure relates generally to predicting the onset of disease or health-related conditions. More particularly, the present disclosure relates to a platform that collects and analyzes heterogenous data streams to be used as inputs for determining behavioral or driving-related anomalies specific to a subject, such as a vehicle driver, which in turn can be used to predict and generate an indication of the onset of disease or health-related conditions.

### DESCRIPTION OF RELATED ART

The prediction of health-related issues, such as the onset of a disease is commonly effectuated based on the identification of pre-determined behaviors or symptoms. Such pre-determined behaviors or symptoms are typically learned from the broader population, rather than being associated with a particular subject. Moreover, such symptoms are typically indicative of a human/physical experience or response.

### BRIEF SUMMARY OF THE DISCLOSURE

In accordance with one embodiment, a computer-implemented method comprises, receiving, from multiple data streams, data related to a vehicle and driver of the vehicle, identifying one or more atypical aspects of the data, and identifying one or more of the atypical aspects of the data that are of interest. The computer-implemented method further comprises determining whether the one or more identified atypical aspects of the data that are of interest are anomalous, and generating and providing a wellness advisory indication to the driver upon a determination that the one or more identified atypical aspects of the data that are of interest are anomalous.

In some embodiments, the data related to the vehicle comprises sensor-generated or sensor-monitored information regarding operational aspects of the vehicle.

In some embodiments, the data related to the driver of the vehicle comprises one or more of data characterizing or reflecting activity of the driver, physiological state of the driver, or health and wellness state of the driver.

In some embodiments, the identification of one or more atypical aspects of the data comprises determining whether aspects of the data fall outside of typical operating or activity parameters defined based on historical instances of the same or similar such aspects of the data. In some embodiments, the identification of one or more of the atypical aspects of the data that are of interest comprises determining whether the aspects of the data fall outside of the typical operating or activity parameters to an extent suggesting that the aspects of the data may be indicative of disease onset. In some embodiments, the aspects of the data comprise one or more of observed actions, events, or features regarding one or more of vehicle behavior or driver behavior. In some embodiments, determining whether the aspects of the data may be indicative of disease onset comprises comparing the aspects of the data to other instances of the one or more observed actions, events, or features. In some embodiments, the determining of whether the one or more identified atypical aspects of the data that are of interest are anomalous is based on one of a magnitude or level of the one or more of the vehicle behavior or the driver behavior exceeding a baseline envelope relative to the other instances of the one or more observed actions, events, or features.

In some embodiments, the computer-implemented method may further comprise correlating the one or more identified atypical aspects of the data that are of interest and are anomalous with one or more disease parameters based on the driver's medical history.

In accordance with one embodiment, a system comprises one or more processors, and a memory storing instructions that when executed, cause the one or more processors to: receive data related to a vehicle and driver of the vehicle; identify one or more atypical aspects of the data; determine whether the one or more atypical aspects of the data are of interest regarding disease onset prediction; determine whether the one or more identified atypical aspects of the data that are of interest are also anomalous; and generate and provide a wellness advisory indication to the driver regarding the one or more atypical aspects of the data that are of interest and are also anomalous.

In some embodiments, the data related to the vehicle comprises sensor-generated or sensor-monitored information regarding operational aspects of the vehicle. In some embodiments, the data related to the driver of the vehicle comprises one or more of data characterizing or reflecting activity of the driver, physiological state of the driver, or health and wellness state of the driver. In some embodiments, the data related to the driver of the vehicle is received from one or more non-vehicular devices used by or associated with the driver of the vehicle.

In some embodiments, the identification of one or more atypical aspects of the data comprises determining whether aspects of the data fall outside of typical operating or activity parameters defined based on historical instances of the same or similar such aspects of the data. In some embodiments, the instructions that cause the one or more processors to identify the one or more of the atypical aspects of the data that are of interest comprises instructions that cause the one or more processors to determine whether the aspects of the data fall outside of the typical operating or activity parameters to an extent suggesting that the aspects of the data may be indicative of disease onset. In some embodiments, the aspects of the data comprise one or more of observed actions, events, or features regarding one or more of vehicle behavior or driver behavior. In some embodiments, the instructions that cause the one or more processors to determine whether the aspects of the data may be indicative of disease onset comprises instructions that cause the one or more processors to compare the aspects of the data to other instances of the one or more observed actions, events, or features. In some embodiments, the determining of whether the one or more identified atypical aspects of the data that are of interest are anomalous is based on one of a magnitude or level of the one or more of the vehicle behavior or the driver behavior exceeding a baseline envelope relative to the other instances of the one or more observed actions, events, or features.

In some embodiments, the instructions further cause the one or more processors to correlate the one or more identified atypical aspects of the data that are of interest and are anomalous with one or more disease parameters based on the driver's medical history.

In some embodiments, the wellness advisory indication is provided by one of a digital twin system or a machine learning model configured to predict a need for the wellness advisory indication.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 illustrates an example integrated health and wellness management system architecture in accordance with one embodiment.

FIG. 2 illustrates an example learning platform in accordance with one embodiment.

FIGS. 3A-3C illustrate example determinations of driving behavior or daily activity extraction, performance assessment, and anomaly detection in accordance with one embodiment.

FIG. 3D is an example architecture of a digital twin system in accordance with one embodiment.

FIG. 4A is a schematic representation of a wellness learning platform implementation of the present disclosure in accordance with one embodiment.

FIG. 4B is a flow chart illustrating example operations for disease onset prediction in accordance with one embodiment.

FIG. 5 is an example computing component that may be used to implement various features of embodiments described in the present disclosure.

The figures are not exhaustive and do not limit the present disclosure to the precise form disclosed.

### DETAILED DESCRIPTION

Disease onset typically refers to the first time that a change or alteration to one's “normal” health status is noted or observed, where the noted change/alteration is characterized by identified signs or symptoms that can be directly attributable to a particular disease or disease process. Predicting disease onset is often made difficult when only a single stream of data points (typically isolated data points) is considered/analyzed. For example, to determine the onset of a heart attack, physical human symptoms may be considered, e.g., chest pain or pressure (angina) that is constant or constantly repeats, feeling of weakness, light-headedness, etc. It can be appreciated that such symptoms that can signal the onset of a heart attack are all conditions that a human physically experiences. This is an example of a single stream of data points.

Moreover, physical human characteristics deemed to be symptoms of the onset of a particular disease tend to be identified from large population-wide observations. For example, particular physical human conditions/behaviors/parameters may only be deemed to be symptoms if some accepted threshold number of people in a particular region(s) (or world-wide) experience those same physical human conditions/behaviors/parameters. However, different persons can have widely varying constitutions (physical and mental), and may exist in or experience a wide variety of environmental conditions that may have an appreciable impact on the onset of a particular disease. Thus, the traditional reliance on population-wide thresholds ignores individual, symptomatic behaviors, instead favoring common, pre-determined behaviors based on, again, population-wide observations. As a result, behavioral cues or parameters shown or exhibited by an individual may be missed or inaccurately/incorrectly characterized. For example, the onset of a heart attack event may occur much more quickly in certain individuals than in other individuals, e.g., an individual who has previously experienced a heart attack is at an increased risk of experiencing another heart attack. Thus, the onset of a heart attack in one individual based on experiencing one instance of angina can occur sooner or later than in another individual also experiencing one instance of angina depending on the individuals' respective histories.

Accordingly, embodiments of the present disclosure are directed to a wellness learning platform for collecting and analyzing heterogenous data streams representative of multiple individualized behavioral and physiological data/parameters or characteristics of users or subjects, such as vehicle drivers. Such parameters can be observed from the users' own actions or physiology/physiological response(s), as well as from “user-adjacent” behaviors or conditions observed, e.g., from the way users operate a vehicle or interact with the users' environment(s). Additionally still, such parameters can be used to train personalized models to predict the onset of disease conditions. Personalized models can be generated using, for example, a digital twin system or machine-learning (ML)/artificial intelligence (AI) mechanisms with which the collection/analytical platform is operatively connected. Indications or notifications regarding suggested remediating actions or instructions in response to identifying some disease onset may be provided to the user.

It should be understood that the embodiments of the present disclosure are rooted in computer technology that overcomes the foregoing and other disadvantages associated with conventional approaches specifically arising in the realm of computer technology. Based on computer technology, the present technology may provide improved techniques for predicting the onset of disease (or other human condition(s)). It should be further understood that the aforementioned aspects of the present technology and the context in which the aforementioned aspects of the present technology operate is such that the amount of data/information that is gleaned, transmitted/received, processed/analyzed, etc. in the manner described herein would not be performable by the human mind. Additionally, the mechanisms for implementing and realizing the embodiments of the present technology, e.g., receiving/processing data from OT sensors/by a computerized wellness learning platform could not be performed by the human mind.

The wellness learning platform operates to collect or obtain characteristics of a human or user state, more generally referred to as human-related data. Such human-related data can include physical behaviors/characteristics or activity of a user along with physiological characteristics, health and wellness characteristics, e.g., body mass index measurements, medical assessment information, lab tests, and the like. The wellness learning platform may also collect user-adjacent characteristics, such as behaviors or characteristics obtained from, e.g., a vehicle operated by a user, that can help define a user's condition via data streams that are not solely human-related. Embodiments are described in the context of vehicle-related data and human-related data, but other user-adjacent contexts are contemplated. For example, the manner in which a user operates a mobile device or the manner in which a user interacts with his/her home may provide data that can be used to predict the onset of a disease. With the recent proliferation of Internet-of-Things (IOT) or more generally (OT) systems and sensors makes obtaining such contextual data possible. As noted above, traditional systems for predicting or analyzing disease onset are limited to singular data streams. The use of data from other types of data streams, e.g., vehicle-related data, can provide additional data points with which the onset of a disease or other condition can be better or more accurately determined.

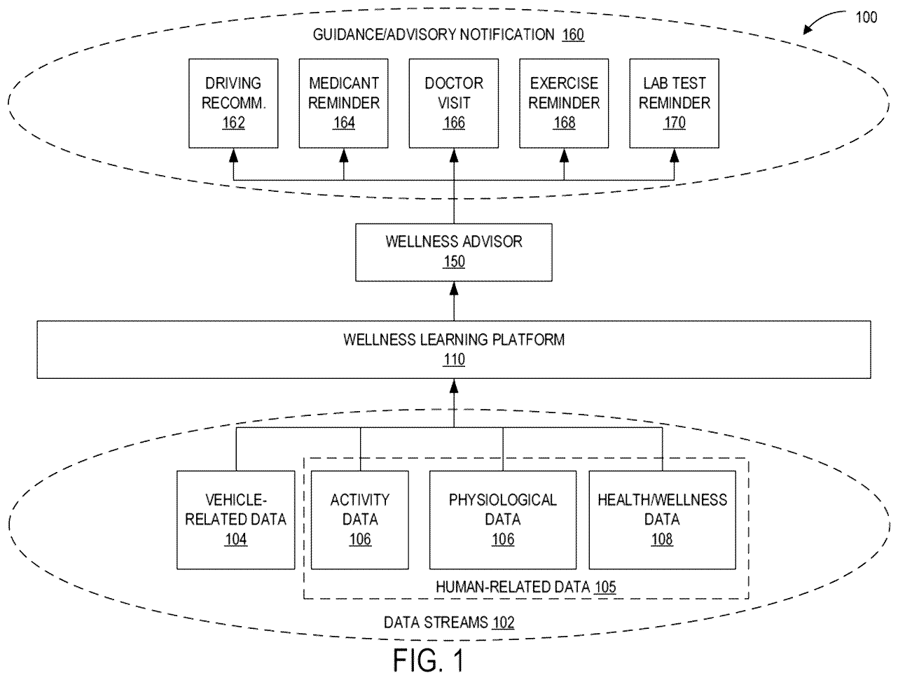

FIG. 1 is a schematic representation of an example system architecture 100 for predicting the onset of disease based on multiple, e.g., heterogenous, data sources or streams. System architecture 100 may include data streams 102. As noted above, embodiments of the present disclosure obtain or receive (human) behavioral data, physiological data, health/wellness data, and vehicle-related data. For example, vehicle-related data 104 may include data or information characterizing operation of a vehicle. Such vehicle-related data 104 may include, but is not limited to sensor-generated or sensor-monitored information regarding operational aspects of a vehicle, e.g., speed of travel, braking frequency, braking magnitude, acceleration events, rate of acceleration, direction of travel, traffic-related information, and so on. Such information or data may be gleaned from a vehicle's controller area network (CAN bus), over which various elements or devices of a vehicle can communicate. In some embodiments, a vehicle that may have the ability, via, e.g., sensors or image/video-capture devices (cameras), to glean data regarding a driver's (or passenger's) conditions, also referred to as user (driver/passenger) behaviors. For example, some vehicles may have pressure sensors for determining an amount of grip being applied to a steering wheel, cameras for detecting facial expressions or cues, physical or physiological reactions or responses to driving scenarios, and the like.

Data streams 102 may further comprise human-related data 105. While vehicle-related data 104 may include or comprise data that can be associated with a human, e.g., a driver/passenger, such as the manner in which a driver operates a vehicle, or reacts to a vehicular scenario, such data, albeit associated with a human actor is data gleaned from or related to vehicle use or operation. While human-related data 105 is related to human behavior(s)/aspects, i.e., activity data 106A and physiological data 106B, such human-related data 105 is typically not associated with a vehicular context/obtained by/from a vehicle, in other words, non-vehicle-related human data. For example, human-related data 105 may comprise human activity or physiological characteristics in a non-vehicular context or that is obtained/gleaned from a non-vehicular mechanism or process.

To the above, and in more recent times, users of wearable devices, such as smartwatches, are able to discern their physiological characteristics at a given point(s) in time. For example, a driver or passenger of a vehicle may be wearing a smartwatch that monitors the driver's/passenger's heart rate, respiration, blood pressure, core body temperature, and the like. Such wearable devices may also be capable of providing activity-related data or characteristics, such as the number of steps a user has taken, typical or historical time for exercise, duration and quality of sleep during a prior period, etc.

Human-related data 105 may further include health/wellness characteristics of a driver or passenger. Health and wellness characteristics data 108 may comprise health or wellness-related events, e.g., bouts of dizziness, fainting episodes, nausea, heart palpitations, excess sweating, and so on.

These data streams 102 may be fed to or obtained/received by wellness learning platform 110. Wellness learning platform 110, as will be described in greater detail below, operates to examine operations of a vehicle and driving behavior (vehicle-related data 104) along with other user-relevant data, i.e., physiological data 106A, activity data 106B, and health/wellness data 108. People generate multiple data streams during daily activities such as driving, walking, jogging, watching TV, sleeping, etc. These data streams can be used as precursors to those peoples' wellness states. For example, when driving, a driver pays proper attention to traffic laws, dynamically changing traffic conditions, etc., to safely and efficiently navigate from point A to point B.

In other daily activities, such as attending to their daily work and chores, people are expected to maintain a certain level of engagement and awareness in order to perform those tasks effectively. However, anomalies in such daily activities can produce clues as to potential health conditions or disease states. One study of drivers with diabetes has shown variability in longitudinal and lateral acceleration when they were traveling at different speeds in highways with hyper- and hypo-glycemic conditions. In another study, drivers with heart arrhythmia tend to exhibit driving errors when symptoms like dizziness, heart palpations, breathing anomalies, etc., were present during high heartrate events.

Therefore, wellness learning platform 110 may examine certain behavioral patterns that correspond to specific disease conditions, as well as, e.g., comorbidity parameters such as, duration and quality of sleep, change in body mass, changes in core body temperature, step counts per day, length of driving time in a given day, number of lane departures, lane centering ability, heard breaking events, hard steering events, number of red light runs, etc.

Different disease conditions will correlate with this data/these parameters, and ML/AI models can be trained to identify the most relevant behavior parameters for each disease condition as precursors to predict the onset of disease or underlying disease conditions. Baseline conditions or thresholds are generated from healthy subjects, while, e.g., daily behavioral observations over time can be evaluated with the baseline conditions to make predictions regarding a person's health and wellness condition.

User activity, affective states, physiology and other data are integrated with their medical and comorbidity conditions into a wellness client or application that generates a digital self of a user, which advisers and instructs the user of engagement of daily activities and routines to continue a healthy life. That is, the wellness client may be a digital wellness twin client that may act as an advisor (wellness advisor 150) that, upon determining a person's health/wellness condition, one or more instructions or indications can be generated and presented to that person. For example, wellness learning platform 110 may determine that a particular driver of a vehicle is at a particular level of risk of some disease (disease severity), and that the driver should take some remediating action(s). Wellness advisor 150 may, based on that determination, generate guidance or advisory notifications 160. The generated guidance or advisory notifications 160 may include, but are not limited to, instructions regarding, e.g., a driving recommendation 152, such as an instruction to cease driving immediately, medicant reminder 154, instruction to visit a doctor 156, instructions to exercise 158, a reminder to conduct lab test 160.

It should be understood that the use of varied data sources/streams and varies types of data to determine disease onset can help avoid inaccuracies. That is, a camera within a vehicle may capture an image of a driver of the vehicle, where the driver is exhibiting excess perspiration, while a steering wheel pressure sensor may sense lighter than usual grip pressure on the steering wheel. Typical systems or methods of determining or predicting disease onset, based on that data alone, may predict the onset of some health condition, e.g., excessive perspiration and weakened physical characteristics may generally suggest one is experiencing a heart attack. However, when multiple/disparate data sources or streams provide data on which to determine whether or not disease onset is occurring, more accurate determinations can be made. That is, the additional data can confirm or verify or contradict a disease onset prediction or determination that is based on the vehicle-related data. Following the above example, receipt of activity data that indicates that user likely just exercised (because the current time is just after the user's usual exercise time), can be analyzed or considered in conjunction with the vehicle-related data. Embodiments of the present disclosure may determine that, taken together, the vehicle-related data and the activity data suggest nothing anomalous or abnormal about the user's condition.

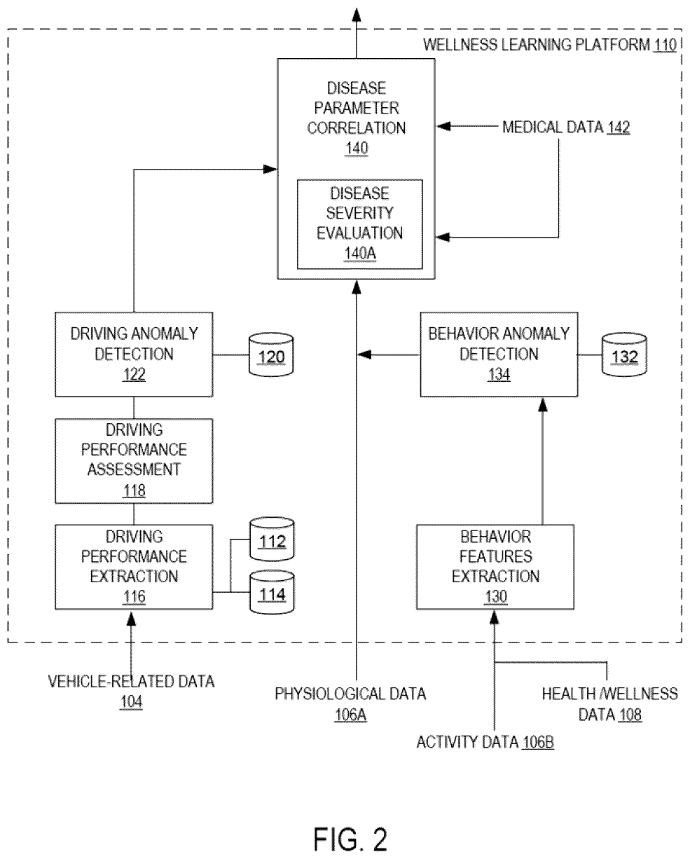

FIG. 2 illustrates example components/operations that may make up/be performed by wellness learning platform 110 in accordance with one embodiment of the present disclosure. As alluded to above, vehicle-related data 104 may be obtained from a vehicle, e.g., data from vehicle sensors, speed profiles, frequency of various maneuvers. Such data can be obtained from a vehicle CAN, and can be stored on internal/in-vehicle storage or remotely in a server/on the cloud. Such vehicle-related data 104 can be received by driving performance extraction component 116. Driving performance extraction component 116 may comprise operations implemented to look for and identify certain vehicle-related features, actions, or events. For example, driving performance extraction component 116 may comprise one or more operations performed by a vehicle's electronic control unit (ECU) or other similar computer processing unit or component. Examples of such actions, features, or events include acceleration events, braking events, and so on.

It should be understood that vehicles do not typically possess much processing power compared to typical computers/workstations, for example. Accordingly, the aforementioned ECU/similar processing unit may not have the processing power/processing resources usually needed to normalize data, such as the vehicle-related data 104—hence the looking for/identifying of particular driving features, activities, or events that can characterize driving and vehicle behavior. Such features, activities, events may be stored and accessed for subsequent correlation with disease parameters (disease parameter correlation component 140) at databases/data repositories such as driver behavior features database 112 and vehicle behavior features database 114. For example, a driver grabbing his/her chest while operating his/her vehicle would be an action/event that applies to, e.g., in-cabin driver behaviors. Accordingly, such an action, event or feature can be extracted/identified from vehicle CAN data as being potentially of interest. As another example, accelerating at a particular rate may suggest an action, event, or feature of interest. In other words, driving performance extraction component 116 may be thought of as a labeling component that “labels” actions, events, or features that are of potential interest from vehicle-related data 104.

Driving performance assessment component 118 may operate to assess the driving performance of a driver (or other passenger). For example, and following the above examples, a driver grabbing his/her chest while operating his/her vehicle would be an action or event that could exceed a standard behavior threshold, e.g., compared to a driver scratching his/her shoulder. As will be described below, actions/events/features may be associated with a “magnitude” or level/severity of an action, event, or feature. The above-described actions, features, or events that have been identified can be compared to other actions, features, or events identified with respect to another vehicle and subject (e.g., driver). Accordingly, “normal” or “standard” actions, events, or features can be determined relative to various vehicles/subjects.

Driving anomaly detection component 122 may operate to determine whether or not the action, event, or feature of interest (determined by driving performance assessment component 118, based on extracted actions, events, features gleaned from vehicle-related data 104) rises to the level of an anomaly (anomalous action, event, or feature) relative to a particular subject's/individual's (e.g., driver) health and wellness characteristics. In this example, a standard action may comprise some determined amount of arm movement relative to the subject's body, and some determined speed at which the arm movement occurs. Following the other example, accelerating at a rate that exceeds, e.g., a typical threshold for acceleration to pass a preceding vehicle, or to get up to speed on an entrance ramp of a freeway, etc. may suggest an action, event, or feature of interest. A database or datastore 120 may comprise baseline information or estimates that can be used as a reference against which observed vehicle/human-related data can be compared to determine if the observed vehicle/human-related data falls within typical norms.

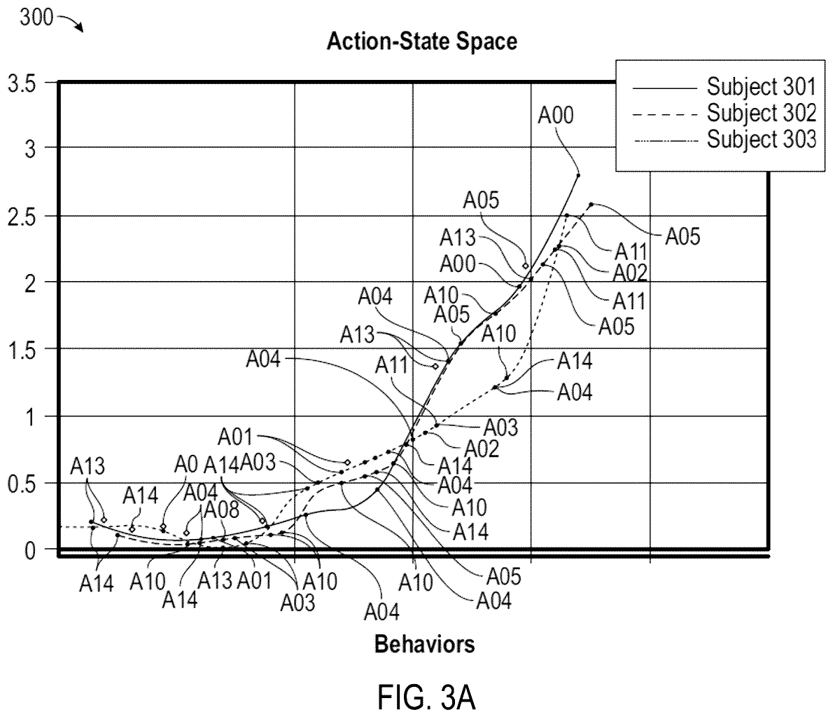

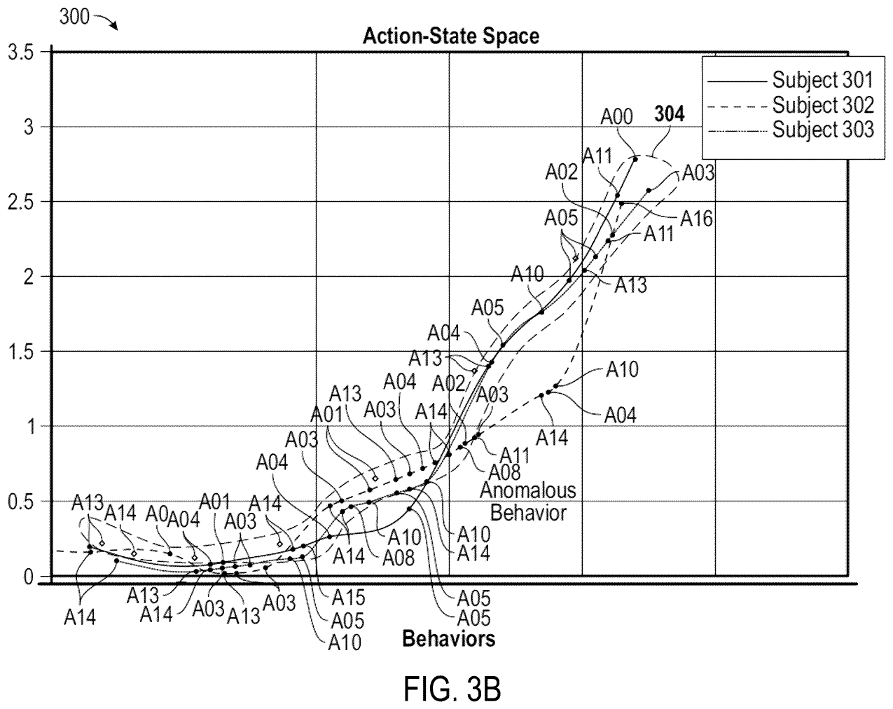

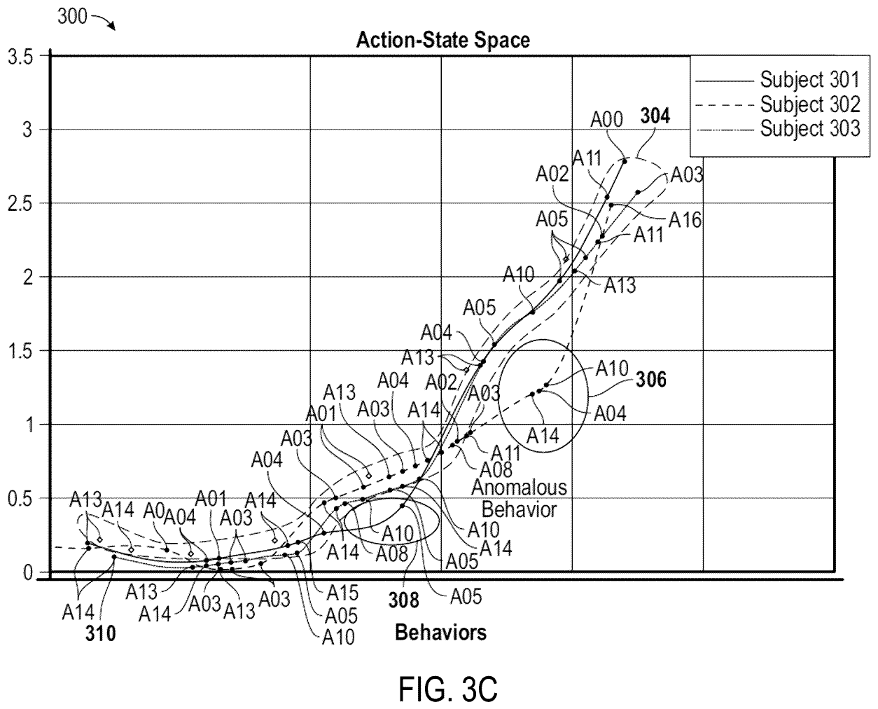

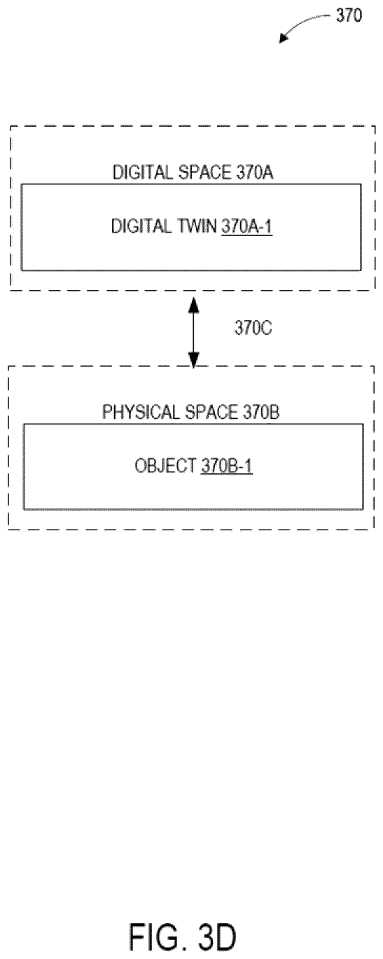

Referring now to FIG. 3A, a graph 300 represents driving, as well as non-driving actions, events, features that have been observed, e.g., an action/state space representation. Graph 300, in this scenario, reflects the performance of particular actions/exhibiting of particular behavior, while operating their respective vehicles or performing daily activities, etc. (subjects 301, 302, 303). As can be appreciated from FIG. 3A, subject 301's behavior is represented as a solid line, subject 302's behavior is represented as a hashed line, and subject 303's behavior is represented as a dotted line. It should be understood that driving performance assessment component 118 need not actually generate graphs as part of its analysis. Rather, graphs such as graph 300 are used for illustrative purposes.

Behavior indicators or identifiers are also reflected in graph 300, some of which, for example's sake, are A13, A14, A01, etc., and recalling that driving performance extraction component 116 acts as, e.g., a labeler, it should be understood that such indicators/identifiers reflect actions, events, or features that could potentially be of interest (for predicting disease or other condition(s) onset). The identifier A13 may refer to head tilting, while A14 may refer to a driver putting hands on a chest, while A01 may refer to hard braking, for example. It should be understood that these are merely examples, and that most any action, event, feature can be tracked/monitored and labeled as described herein.

Graph 300 reflects instances or occurrences of actions, events, features, in particular their respective magnitudes (on the y-axis), while the “timing” of the occurrences/instances is reflected along the x-axis. It should be understand that the timing of an occurrence per se, is not necessarily critical in various embodiments-hence the lack of measure along the x-axis. Rather, it may be sufficient for purposes of disease/condition onset prediction, to know, e.g., an event occurred within a monitored or sampled span of time or during traversal of a particular path, or the number of events that occurred, relative frequency, etc.

Referring to FIG. 3B, it can be appreciated that based on baselines/baseline estimates of typical behaviors stored in baseline database 120, certain actions or behaviors may fall within those baselines, while certain actions or behaviors may fall outside of those baselines. In this example, nearly all of subject 301's and subject 303's behaviors are commensurate with the baseline data, i.e., the majority of subject 301's and subject 303's behavior magnitudes are encompassed by baseline behavior envelope 304.

Referring to FIG. 3C, it can be appreciated that any behaviors falling outside of baseline envelope 304 may be anomalous occurrences (e.g., anomalous in terms of behavior magnitude). It should be appreciated that embodiments may determine anomalous behaviors based on a magnitude/level of behavior that exceeds the baseline envelope 304 by a given amount. For example, some behaviors of subject 303 such as the first occurrence of a driver putting hands on a chest, indicated by the first A14 of graph 300. However, while the baseline envelope 304 suggests normal behavior when the magnitude of an A14 action is between approximately between 0.22 and 0.375 along a “first portion” of travel, an A14 action (labeled 310 in FIG. 3C) with a magnitude of approximately 0.2 is not deemed to be an instance of anomalous behavior. However, in the case of the occurrence of A13, A14, and A01 events (grouped/labeled 306), such behaviors exceed the baseline envelope 304 by enough that such behaviors may be considered to be anomalous behavior. The same holds true of the occurrence of the A01 event noted as anomalous behavior 308.

It should be understood that implementation of such techniques for determining normal versus anomalous behavior can be realized by driving performance extraction component 116, driving performance assessment component 118, and driving anomaly detection 122.

Referring back to FIG. 2, it can be appreciated that human-related data 105 (more particularly, physiological data 106A, activity data 106B, and health/wellness data 108) may be similarly considered and analyzed to ultimately predict, e.g., disease onset. Physiological data 106A can comprise, but is not limited to, e.g., data regarding heart rate, respiratory function, oxygen saturation (SpO2), blood pressure, body temperature, and so on. Such data can be transmitted to/received by disease parameter correlation component 140, e.g., from wearable devices/sensors, and can be used, similar to vehicle-related data 104, to characterize a subject's physiological state. It should be appreciated that such parameters or measures are “direct” indicators of health/wellness, and thus can be utilized by disease parameter correlation component 140 as-is (without additional extraction, assessment, detection).

Still other human-related data 105, e.g., activity data 106B (such as step count, exercise duration, sleep quality, etc.), and health/wellness data 108 (e.g., dizziness events, fainting events, occurrences of nausea, excess sweating, heart palpitations, and so on) can be leveraged, again like vehicle-related data 104. Activity data 106B and health/wellness data 108 can be collected using devices associated with the subject, e.g., wearable and non-wearable devices, such as smartwatches, smartphones, vitals-monitoring devices such as continuous glucose monitors, cardiograms, active and passive cameras, Bluetooth-enabled devices, etc. These data streams can be recorded during/while a subject performs or goes about his/her daily activities. Also, historical data, such as vitals data can be gathered from electronic medical/health records, hospital visits, and lab tests. Driving behaviors and performance data can be collected from driver monitor cameras, CAN, IMU, accelerometers, GPU units embedded in the vehicles, and so on Behavior features extraction component 130 may perform similar operations as those described above regarding driving performance extraction component 116 to extract human-related actions, events, features. Similar to driving anomaly detection component 122, behavior anomaly detection component 134 (based on behavior baseline information stored in datastore 132, which in turn is similar to driving baseline information 120) may determine if anomalous activities or health/wellness events exist in the received human-related data 106B/108.

Regarding the behavior analysis described herein, the number of datasets that are collected/accumulated will typically be about 20-25 datasets for each subject from their daily activities, but can vary depending on environmental conditions, particular behaviors, depending on the subject's own characteristics, historical data, etc. For example, observation of activity levels such as the number of workout sessions a subject undergoes per day/week, the number of steps accumulated per day/week, duration of sleep, etc. are analyzed. Similarly, in the driving context, average speed for a given route within a day/week, number of safety critical incidents per trip per day/week, etc. are analyzed during an observation period. If a subject demonstrates consistent behaviors (in daily activities and driving) during the observation period, their baseline graphs are generated. If some of their behaviors were inconsistent, the observation period may extend to accumulate more data points to generate more accurate baselines.]

Now that anomalous behavior (actions, events, features) have been identified from vehicle-related data 104 and human-related data 105, disease parameter correlation component 140 operates to associate driving or behavior anomalies with a subject's actual medical history/records. In this way, disease parameter correlation component 140 is able to both (a) identify what actions, events, features are foreshadowing a condition/disease that a subject is known to have, and (b) determine the severity of the condition or disease (140A).

That is, upon receiving anomalous driving or behavior data, disease parameter correlation component 140 can associate an event, such as the subject grabbing his/her chest or repeated hard braking with a condition of the subject, e.g., atrial fibrillation, that is known by virtue of medical data 142 regarding that particular subject (e.g., lab/medical records, body mass index, and so on). As alluded to above, embodiments of the present application are directed to more accurately predicting the onset of disease or other condition because individualized data is considered and analyzed, rather than relying on population-wide data. Again, non-individualized data or parameters that can be disease onset predictors vary between subjects. Because embodiments of the present disclosure leverage a subject's own medical history/information, disease onset predictions made by embodiments are specific to that subject, and thus, more accurate. Additionally, the severity of that subject's disease/condition can be determined. For example, the number of driving or behavioral anomalies detected, or the relative frequency of occurrences of such anomalies may suggest that the onset of a disease, such as atrial fibrillation is more/less likely to occur or likely to occur sooner or later, etc.

As discussed previously, wellness advisor 150 may be implemented as a digital twin system. FIG. 3D illustrates an example digital twin system 370 comprising: a digital space 370A comprising a digital twin 370A-1 (which may be an embodiment of wellness advisor 150 (or implemented therein) of FIG. 1) representative of an object of interest 370B-1 (for example, the driver of a vehicle); a physical space 370B associated with objects of interest; and a communications layer 370C that enables communications between the physical and digital spaces 370A/370B. It should be understood that a digital twin can refer to some representation, e.g., virtual, of an object, system, or other entity, again, in this case, a driver of a vehicle or subject.

A digital twin acts as a digital counterpart or model to some physical object or process, such a vehicle driver. The physical object/process can be outfitted with or monitored using sensors that generate data regarding various aspects of the physical object/process, e.g., the performance of the physical object. In this case, such sensors may be wearable sensors providing user activity parameters, vehicle sensors providing driving-related parameters, etc. This generated data (in this case, from the learning platform, e.g., wellness platform 110), can be relayed to a processor or other computing system which may then apply the data to the digital twin. Thereafter, the digital twin or model can be used to run simulations, study performance, generate possible improvements, and so on. In this case, the digital twin 370A-1 can be used to determine what the driver/subject should do upon wellness platform 110 predicting the onset of a disease or other condition. Thus, digital twin 370A-1 can generate guidance or advice that may be sent to or other presented to the corresponding user, i.e., object 370B-1 regarding one or more reminders, actions, etc. that the user may take to prevent or at least mitigate the onset of disease (that was correlated to vehicle-related data 104/human-related data 105).

As illustrated in FIG. 3D, physical space 370B, in which actors, such as the object of interest 370B-1, logically “reside.” Sampling and actuation processes or procedures may occur in physical space 370B. That is, sensors or devices capable of monitoring actors detect the dynamic status of an actor, any ongoing operations, or any event occurrences associated with the actor or impacting the actor. This sensor data or information, e.g., data samples or measurements, can be aggregated for transmission to digital space 370A. Such data/information can be analyzed or processed by digital space 370A vis-à-vis the respective digital twin to which the data/information apply. Processing/analyzing the data can comprise different operations, but will ultimately produce some output(s) from a mechanism, such as a machine learning algorithm, a resulting perception, etc. that can be used to guide or instruct the subject.

It should be understood that the use of a digital twin system is one contemplated implementation. However, other embodiments may leverage ML/AI to infer or predict guidance/advice. In some embodiments, wellness advisory 150 may comprise certain ML/AI models generated to predict the need for particular aforementioned guidance/advisory notifications 160. For example, ML/AI algorithms may be developed for predicting what guidance or advise is to be given based on the occurrence or detection of disease onset. Accordingly, the same/similar vehicle-related data 104 and human-related data 105 can be used to train such ML/AI algorithms, resulting in the advice-predictive ML/AI models.

Wellness learning platform 110 too, may be implemented in various ways, e.g., as a scalable client-server architecture, in secure cloud servers or stand-alone, isolated servers. These servers can typically range from distributed mainframes to localized computers in domestic living environments. The clients can be implemented in portable wearable devices where users carry in daily activities or in vehicle cabins, airplane cockpits, and other machine operator environments where a user's activities, characteristics, performance is measurable. In some embodiments aspects of wellness learning platform 110 may be distributed between a server and in-vehicle processor(s)/computing systems.

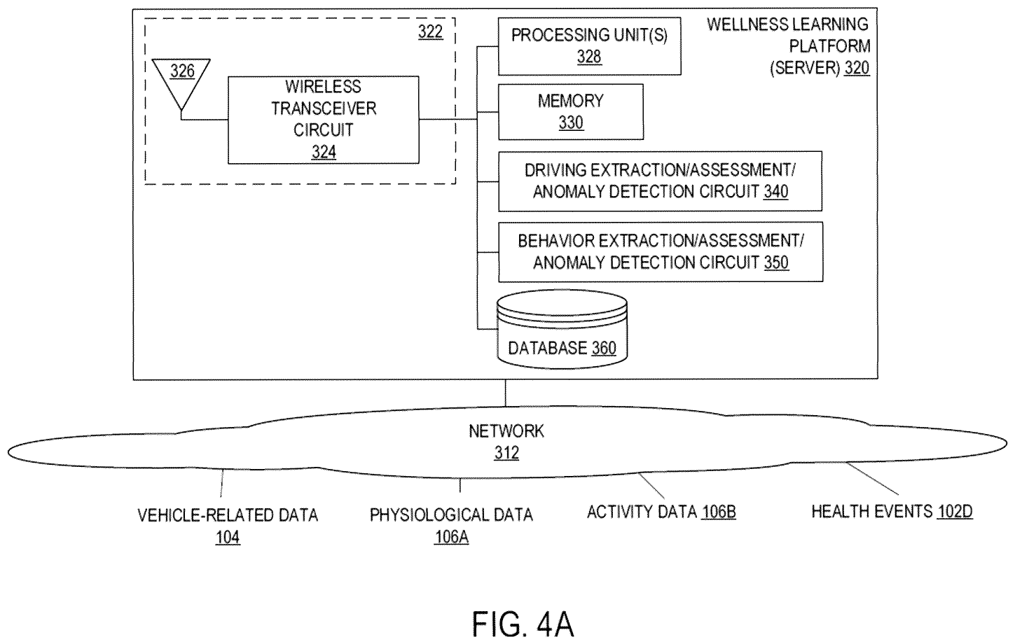

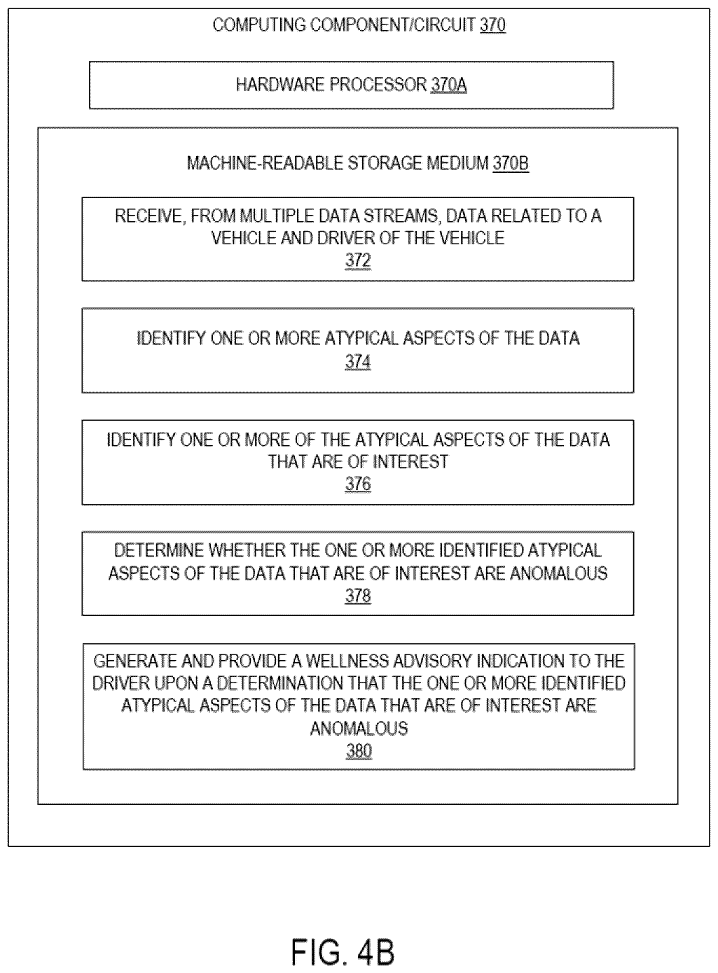

FIG. 4A illustrates an example embodiment of wellness learning platform 110, as a server 320. Server 320 in this example, includes: a communication circuit 322; a driving extraction/assessment/anomaly detection circuit 340 (an embodiment of components 116, 118, and 122 (FIG. 2); a behavior extraction/assessment/anomaly detection circuit 340 (an embodiment of components 130 and 134 (FIG. 2); a database 360 (which may embody one or more of datastores 112, 114, 120, and 132; a processing unit(s) 328; and memory 330. Components of server 320 are illustrated as communicating with each other via a data bus, although other communication interfaces can be included.

Processing unit(s) 328 can include a GPU, CPU, microprocessor, or any other suitable processing system. Memory 330 may include one or more various forms of memory or data storage (e.g., flash, RAM, etc.) that may be used to store the calibration parameters, images (analysis or historic), point parameters, instructions and variables for processing unit(s) 328 as well as any other suitable information. Memory 330, can be made up of one or more modules of one or more different types of memory, and may be configured to store data and other information as well as operational instructions that may be used by processing unit(s) 328 to assist/effectuate server 320's functionality.

Although the example of FIG. 4A is illustrated using processor and memory circuitry, as described below with reference to circuits disclosed herein, either one or both of driving extraction/assessment/anomaly detection circuit 340 and behavior extraction/assessment/anomaly detection circuit 350 can be implemented utilizing any form of circuitry including, for example, hardware, software, or a combination thereof. By way of further example, one or more processors, controllers, ASICs, PLAS, PALs, CPLDs, FPGAs, logical components, software routines or other mechanisms might be implemented to make up driving extraction/assessment/anomaly detection circuit 340 and behavior extraction/assessment/anomaly detection circuit 350.

Communication circuit 322 may comprise a wireless transceiver circuit 324 with an associated antenna 326. Wireless transceiver circuit 324 can include a transmitter and a receiver (not shown) to allow wireless communications via any of a number of communication protocols such as, for example, WiFi, Bluetooth, near field communications (NFC), Zigbee, and any of a number of other wireless communication protocols whether standardized, proprietary, open, point-to-point, networked or otherwise. Antenna 326 is coupled to wireless transceiver circuit 324 and is used by wireless transceiver circuit 324 to transmit radio signals wirelessly to wireless equipment with which it is connected and to receive radio signals as well. These RF signals can include information of almost any sort that is sent or received by driving extraction/assessment/anomaly detection circuit 340 and behavior extraction/assessment/anomaly detection circuit 350 to/from other entities such as sensors, vehicle systems, wearable devices, and so on. As illustrated in FIG. 4A, for example, vehicle-related data 104, physiological data 106A, activity data 106B, and heath/wellness data 108 may be transmitted to server 320 via a network 312, which may be any appropriate communications network.

FIG. 4B is an example computing component/circuit 370 that may be used to implement various features of driving or behavior extraction/assessment/anomaly detection in accordance with one example of the disclosed technology. Computing component 340 may be, for example, a computer, a controller, or any other similar computing component capable of processing data. In the example implementation of FIG. 4B, the computing component 370 includes a hardware processor 370A, and machine-readable storage medium 370B.

Hardware processor 370A may be one or more central processing units (CPUs), semiconductor-based microprocessors, and/or other hardware devices suitable for retrieval and execution of instructions stored in machine-readable storage medium 340B. Hardware processor 370A may fetch, decode, and execute instructions, such as instructions 372-380, to assess driving and detect anomalies associated with a user's operation of a vehicle. As discussed above, conventional wellness systems or methodologies rely on singular data streams, whereas in accordance with some embodiments, user-adjacent data, such as vehicle-related parameters may be obtained and associated with user data, such as human-related parameters, to predict disease onset. As an alternative or in addition to retrieving and executing instructions, hardware processor 370A may include one or more electronic circuits that include electronic components for performing the functionality of one or more instructions, such as a field programmable gate array (FPGA), application specific integrated circuit (ASIC), or other electronic circuits.

A machine-readable storage medium, such as machine-readable storage medium 370B, may be any electronic, magnetic, optical, or other physical storage device that contains or stores executable instructions. Thus, machine-readable storage medium 370B may be, for example, Random Access Memory (RAM), non-volatile RAM (NVRAM), an Electrically Erasable Programmable Read-Only Memory (EEPROM), a storage device, an optical disc, and the like. In some examples, machine-readable storage medium 604 may be a non-transitory storage medium, where the term “non-transitory” does not encompass transitory propagating signals. As described in detail below, machine-readable storage medium 370B may be encoded with executable instructions, for example, instructions 372-380.

Hardware processor 370A may execute instruction 372 to receive, from multiple data streams, data related to a vehicle and driver of the vehicle. As previously described, embodiments of the present disclosure leverage various types/sources of data, e.g., vehicle-related data (operational states/conditions), and human-related data (physiological measurements/states, activities, events, health/wellness characteristics) to increase accuracy in disease onset prediction. Again, vehicles may comprise sensors that monitor vehicle operation/state and can relay data. The same is true of wearable devices, communication devices, e.g., smartphones, that may have health/wellness functions that can provide driving/behavioral characteristics.

Hardware processor 370A may execute instruction 374 to identify one or more atypical aspects of the data. As described above, data can be received from a variety of sources, and such data can be analyzed to determine or identify actions, events, or features that occur, e.g., while a subject is driving a vehicle along a particular path. Some such actions, features, events may be typical (speeding up on a freeway entrance ramp, repeated braking in traffic), while others may not be typical (excessive sweatiness, inconsistent acceleration events). Regardless, depending on a particular vehicle/traffic conditions, or subject (driver), for example, even though an action or event or feature may be atypical, it may not necessarily be a disease onset indicator. Some such actions, events, features regarding vehicle operation, driver behavior during vehicle operation, other health/wellness states or characteristics such vital signs, activity data, such as exercise time and frequency may fall outside “normal” operating or activity parameters.

Accordingly, hardware processor 370A may execute instruction 376 to identify one or more the atypical aspects of the data that are of interest. In other words, there may be certain observed actions, events, features that exceed normal operation to the extent that they are of interest, in that they may be indicators of disease onset. As described above with respect to, e.g., FIGS. 2 and 3A-3C, wellness platform 110 may comprise components that can analyze multiple vehicles/drivers that, e.g., travel along the same or similar path, perhaps at or near the same time(s) by comparing relative instances of those actions, events, or features to determine which may be disease onset predictors.

Hardware processor 370A may execute instruction 378 to determine whether the one or more identified atypical aspects of the data that are of interest are anomalous. Given the received data and the analysis of that data, certain actions, events, or features may fall outside typical or normal parameters that they can be deemed to be anomalous relative to other observed data. As described above, anomalous behaviors can be determined based on a magnitude/level of behavior that exceeds a baseline envelope relative to the observed data.

Once anomalous data has been identified, hardware processor 370A may execute instruction 380 to generate and provide a wellness advisory indication to the driver/subject upon a determination that the one or more identified atypical aspects of the data that are of interest are anomalous. That is, such anomalous data can be correlated with disease parameters based on a subject's own medical history, for example, again resulting in better accuracy of predicting disease onset. In other words, certain anomalous actions that may have been identified from the received data may not be disease onset predictors given a subject's medical history, while for other subjects, the same anomalous actions may indeed be indicative of disease onset. Moreover, the severity of the disease/disease onset can also be determined based on a subject's particular health/wellness characteristics. This severity determination, along with the aforementioned correlation of anomalous action/event/feature data to disease onset allows embodiments of the present application to generate and provide appropriate warnings or alerts or instructions to avoid or mitigate disease onset (or avoid collateral issues, e.g., accident prevention by instructing a driver to stop driving or to see a medical professional, etc.

It should be noted that the terms “optimize,” “optimal” and the like as used herein can be used to mean making or achieving performance as effective or perfect as possible. However, as one of ordinary skill in the art reading this document will recognize, perfection cannot always be achieved. Accordingly, these terms can also encompass making or achieving performance as good or effective as possible or practical under the given circumstances, or making or achieving performance better than that which can be achieved with other settings or parameters

It should be noted that the terms “approximately” and “about” used throughout this disclosure, including the claims, are used to describe and account for small deviations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%

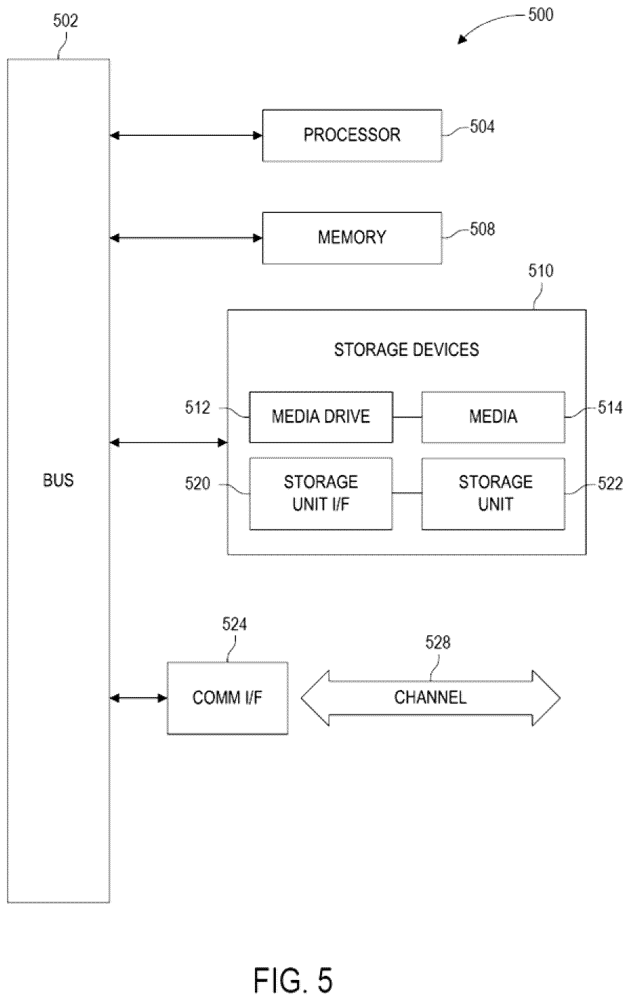

As used herein, the terms circuit and component might describe a given unit of functionality that can be performed in accordance with one or more embodiments of the present application. As used herein, a component might be implemented utilizing any form of hardware, software, or a combination thereof. For example, one or more processors, controllers, ASICs, PLAS, PALs, CPLDs, FPGAs, logical components, software routines or other mechanisms might be implemented to make up a component. Various components described herein may be implemented as discrete components or described functions and features can be shared in part or in total among one or more components. In other words, as would be apparent to one of ordinary skill in the art after reading this description, the various features and functionality described herein may be implemented in any given application. They can be implemented in one or more separate or shared components in various combinations and permutations. Although various features or functional elements may be individually described or claimed as separate components, it should be understood that these features/functionality can be shared among one or more common software and hardware elements. Such a description shall not require or imply that separate hardware or software components are used to implement such features or functionality.

Where components are implemented in whole or in part using software, these software elements can be implemented to operate with a computing or processing component capable of carrying out the functionality described with respect thereto. One such example computing component is shown in FIG. 5. Various embodiments are described in terms of this example-computing component 500. After reading this description, it will become apparent to a person skilled in the relevant art how to implement the application using other computing components or architectures.

Referring now to FIG. 5, computing component 500 may represent, for example, computing or processing capabilities found within a self-adjusting display, desktop, laptop, notebook, and tablet computers. They may be found in hand-held computing devices (tablets, PDA's, smart phones, cell phones, palmtops, etc.). They may be found in workstations or other devices with displays, servers, or any other type of special-purpose or general-purpose computing devices as may be desirable or appropriate for a given application or environment. Computing component 500 might also represent computing capabilities embedded within or otherwise available to a given device. For example, a computing component might be found in other electronic devices such as, for example, portable computing devices, and other electronic devices that might include some form of processing capability.

Computing component 500 might include, for example, one or more processors, controllers, control components, or other processing devices. This can include a processor, and/or any one or more of the components making up the vehicle data gathering circuit 310 or any processing components/elements of FIG. 4, for example. Processor 504 might be implemented using a general-purpose or special-purpose processing engine such as, for example, a microprocessor, controller, or other control logic. Processor 504 may be connected to a bus 502. However, any communication medium can be used to facilitate interaction with other components of computing component 500 or to communicate externally.

Computing component 500 might also include one or more memory components, simply referred to herein as main memory 508. For example, random access memory (RAM) or other dynamic memory, might be used for storing information and instructions to be executed by processor 504. Main memory 508 might also be used for storing temporary variables or other intermediate information during execution of instructions to be executed by processor 504. Computing component 500 might likewise include a read only memory (“ROM”) or other static storage device coupled to bus 502 for storing static information and instructions for processor 504.

The computing component 500 might also include one or more various forms of information storage mechanism 510, which might include, for example, a media drive 512 and a storage unit interface 520. The media drive 512 might include a drive or other mechanism to support fixed or removable storage media 514. For example, a hard disk drive, a solid-state drive, a magnetic tape drive, an optical drive, a compact disc (CD) or digital video disc (DVD) drive (R or RW), or other removable or fixed media drive might be provided. Storage media 514 might include, for example, a hard disk, an integrated circuit assembly, magnetic tape, cartridge, optical disk, a CD or DVD. Storage media 514 may be any other fixed or removable medium that is read by, written to or accessed by media drive 512. As these examples illustrate, the storage media 514 can include a computer usable storage medium having stored therein computer software or data.

In alternative embodiments, information storage mechanism 510 might include other similar instrumentalities for allowing computer programs or other instructions or data to be loaded into computing component 500. Such instrumentalities might include, for example, a fixed or removable storage unit 522 and an interface 520. Examples of such storage units 522 and interfaces 520 can include a program cartridge and cartridge interface, a removable memory (for example, a flash memory or other removable memory component) and memory slot. Other examples may include a PCMCIA slot and card, and other fixed or removable storage units 522 and interfaces 520 that allow software and data to be transferred from storage unit 522 to computing component 500.

Computing component 500 might also include a communications interface 524. Communications interface 524 might be used to allow software and data to be transferred between computing component 500 and external devices. Examples of communications interface 524 might include a modem or softmodem, a network interface (such as Ethernet, network interface card, IEEE 802.XX or other interface). Other examples include a communications port (such as for example, a USB port, IR port, RS232 port Bluetooth® interface, or other port), or other communications interface. Software/data transferred via communications interface 524 may be carried on signals, which can be electronic, electromagnetic (which includes optical) or other signals capable of being exchanged by a given communications interface 524. These signals might be provided to communications interface 524 via a channel 528. Channel 528 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media. Such media may be, e.g., memory 508, storage unit 520, media 514, and channel 528. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 500 to perform features or functions of the present application as discussed herein.

It should be understood that the various features, aspects and functionality described in one or more of the individual embodiments are not limited in their applicability to the particular embodiment with which they are described. Instead, they can be applied, alone or in various combinations, to one or more other embodiments, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A computer-implemented method, comprising:
receiving, from multiple data streams, data related to a vehicle and driver of the vehicle;
identifying one or more atypical aspects of the data;
identifying one or more of the atypical aspects of the data that are of interest;
determining whether the one or more identified atypical aspects of the data that are of interest are anomalous; and
generating and providing a wellness advisory indication to the driver upon a determination that the one or more identified atypical aspects of the data that are of interest are anomalous.

2. The computer-implemented method of claim 1, wherein the data related to the vehicle comprises sensor-generated or sensor-monitored information regarding operational aspects of the vehicle.

3. The computer-implemented method of claim 1, wherein the data related to the driver of the vehicle comprises one or more of data characterizing or reflecting activity of the driver, physiological state of the driver, or health and wellness state of the driver.

4. The computer-implemented method of claim 1, wherein the identification of one or more atypical aspects of the data comprises determining whether aspects of the data fall outside of typical operating or activity parameters defined based on historical instances of the same or similar such aspects of the data.

5. The computer-implemented method of claim 4, wherein the identification of one or more of the atypical aspects of the data that are of interest comprises determining whether the aspects of the data fall outside of the typical operating or activity parameters to an extent suggesting that the aspects of the data may be indicative of disease onset.

6. The computer-implemented method of claim 5, wherein the aspects of the data comprise one or more of observed actions, events, or features regarding one or more of vehicle behavior or driver behavior.

7. The computer-implemented method of claim 6, wherein determining whether the aspects of the data may be indicative of disease onset comprises comparing the aspects of the data to other instances of the one or more observed actions, events, or features.

8. The computer-implemented method of claim 7, wherein the determining of whether the one or more identified atypical aspects of the data that are of interest are anomalous is based on one of a magnitude or level of the one or more of the vehicle behavior or the driver behavior exceeding a baseline envelope relative to the other instances of the one or more observed actions, events, or features.

9. The computer-implemented method of claim 1, further comprising correlating the one or more identified atypical aspects of the data that are of interest and are anomalous with one or more disease parameters based on the driver's medical history.

10. A system, comprising:
one or more processors; and
a memory storing instructions that when executed, cause the one or more processors to:
receive data related to a vehicle and driver of the vehicle;
identify one or more atypical aspects of the data;
determine whether the one or more atypical aspects of the data are of interest regarding disease onset prediction;
determine whether the one or more identified atypical aspects of the data that are of interest are also anomalous;
generate and provide a wellness advisory indication to the driver regarding the one or more atypical aspects of the data that are of interest and are also anomalous.

11. The system of claim 10, wherein the data related to the vehicle comprises sensor-generated or sensor-monitored information regarding operational aspects of the vehicle.

12. The system of claim 10, wherein the data related to the driver of the vehicle comprises one or more of data characterizing or reflecting activity of the driver, physiological state of the driver, or health and wellness state of the driver.

13. The system of claim 12, wherein the data related to the driver of the vehicle is received from one or more non-vehicular devices used by or associated with the driver of the vehicle.

14. The system of claim 10, wherein the identification of one or more atypical aspects of the data comprises determining whether aspects of the data fall outside of typical operating or activity parameters defined based on historical instances of the same or similar such aspects of the data.

15. The system of claim 14, wherein the instructions that cause the one or more processors to identify the one or more of the atypical aspects of the data that are of interest comprises instructions that cause the one or more processors to determine whether the aspects of the data fall outside of the typical operating or activity parameters to an extent suggesting that the aspects of the data may be indicative of disease onset.

16. The system of claim 15, wherein the aspects of the data comprise one or more of observed actions, events, or features regarding one or more of vehicle behavior or driver behavior.

17. The system of claim 16, wherein the instructions that cause the one or more processors to determine whether the aspects of the data may be indicative of disease onset comprises instructions that cause the one or more processors to compare the aspects of the data to other instances of the one or more observed actions, events, or features.

18. The system of claim 17, wherein the determining of whether the one or more identified atypical aspects of the data that are of interest are anomalous is based on one of a magnitude or level of the one or more of the vehicle behavior or the driver behavior exceeding a baseline envelope relative to the other instances of the one or more observed actions, events, or features.

19. The system of claim 1, wherein the instructions further cause the one or more processors to correlate the one or more identified atypical aspects of the data that are of interest and are anomalous with one or more disease parameters based on the driver's medical history.

20. The system of claim 1, wherein the wellness advisory indication is provided by one of a digital twin system or a machine learning model configured to predict a need for the wellness advisory indication.
